(19) United States
(12) Patent Application Publication
(10) Pub. No.: US 2024/0016868 A1
(43) Pub. Date: Jan. 18, 2024

(54) Color Coded Cannabis Variety Pack and Directive of Use

(71) Applicants: Gregory King, Pryor, OK (US); Stacy King, Pryor, OK (US)

(72) Inventors: Gregory King, Pryor, OK (US); Stacy King, Pryor, OK (US)

(21) Appl. No.: 17/867,604

(22) Filed: Jul. 18, 2022

(51) Int. Cl. A61K 36/185; A24D 1/18; A23L 33/105; B65D 85/08; A61K 45/06

(52) U.S. Cl. CPC A61K 36/185; A24D 1/18; A23L 33/105; B65D 85/08; A61K 45/06; B65D 2203/02

(57) ABSTRACT

A novel device and method of use featuring color-coded or otherwise distinguishable cannabis containing products wherein the color or distinguishable feature identifies the strain of cannabis contained in the item wherein users make their selection according to the type of strain desired and what type of effect it may have upon the user.

### CROSS-REFERENCE TO RELATED APPLICATIONS

This application claims priority U.S. Patent Provisional Application No. 63/276,289, “Color Coded Cannabis Variety Pack and Directive of Use” filed on Nov. 5, 2021.

### BACKGROUND

### Field of Use

The subject matter in this patent application is to be used for directing users to what strain of cannabis within the packaging to consume depending upon the physiological or psychological symptom to alleviate. The package would include various strains of marijuana or cannabis in at least one consumable form.

The preferred embodiment of the device allows for the use of various strains of cannabis in order to alleviate or supplement physiological or psychological conditions.

The preferred embodiment of the device allows for identification of each strain. Each strain is color coded with a reference table to identify each strain, potency of THC or other cannabinoid, terpene amount, and directive toward which strain is best for particular symptoms or ailments.

The preferred embodiment of the device mitigates the confusion of the user trying to identify which strain to consume, particularly within the particular package the user is in possession of.

The device consists of packaging containing color coded strains of at least one consumable form. The device consists of packaging featuring a table referencing the color coded strains within. Functions of the device and method of use include: color coded consumable strains of cannabis; a reference table defining said color coded strains within; said reference table comprising strain name, type of strain, potency of cannabinoid, terpene potency, batch number of strain, and what the specific strain can be useful for.

The subject matter of this patent application is meant to direct users to consume a specific strain of cannabinoid containing product to achieve a desired physiological or psychological reaction or perhaps both.

### Background of the Prior Art

Cannabis, including cannabinoids THC, CBD and others have been shown to alleviate and even treat many physiological and psychological symptoms and afflictions. Within the plant family of Cannabaceae, commonly called Cannabis, there are two main known subspecies Cannabis Sativa and Cannabis Indica. These two main subspecies are then further cultivated by specific traits to create strains that can be used to provide users a type of physiological and/or psychological reaction and can even alleviate and mitigate the many physiological and psychological symptoms that users are experiencing. Some of these symptoms are insomnia, nausea, anxiety, depression, attention disorders, pain, inflammation, and many others.

The current issue with the existing technology is that many users do not know which strains to select for the desired effect. Existing pre-rolled marijuana cigarettes and other products are sold individually according to a specific strain or even simply labeled as subspecies indica or sativa or a hybrid of the two. Consumers are confused as to which one to take and can often get them mixed up. If the label of the product is lost or falls off, then the consumer might not know what strain or subspecies it is they are consuming. Simple dividers within packaging also are likely to fall out and different strains are unidentifiable. Nothing is currently known nor exists on the market to prevent such problems.

### DESCRIPTION

Disclosed herein is a multi-dimensional product that enables users to be able to select and know which strain of cannabis they are consuming and gives them a directive as to which type of physiological and/or psychological effect they wish to achieve.

All elements of the device can be modified to encompass all different available strains of cannabis or other psychoactive products.

The preferred embodiment of the device comprises: at least two or more strains of cannabis or other psychoactive substance; color-specific or individually-identifiable wrapping or sub packaging specific to each included strain; labeling on the outer packaging or container to provide a reference for each said two or more strains contained within said color specific sub packaging.

The preferred embodiment of the device comprises different strains of cannabis contained within packaging which is a rolling paper secured by a colored cone.

As used herein, the term “cannabis” is meant to encompass any raw or processed form of a substance containing any cannabinoid.

As used herein, the term “cannabinoid” is meant to encompass any of a group of closely related compounds which include cannabidiol and the active constituents of cannabis.

As used herein, the term “pre-roll” is meant to encompass a filled cigarette or joint that is consumer ready to use.

As used herein, the term “cones” is meant to encompass the material or packaging the cannabis is contained within for a pre-roll. Other similar terms include but are not limited to “blunt” or “joint.”

As used herein, the term, “package” is meant to encompass the outer package within which the pre-rolls or other products are contained as well as to which the label is affixed to.

As used herein, the term “physiological” is meant to encompass the way in which a living organism functions.

As used herein, the term “psychological” is meant to encompass that which relates to the mental and emotional state of a person.

As used herein, the term “strain” is meant to encompass either pure or hybrid varieties of the plant genus Cannabis, which encompasses the species C. sativa, C. indica, or C. ruderalis.

As used herein, the term “potency” is meant to encompass the concentration of active ingredient within a product.

As used herein, the term “terpene” is meant to encompass the aromatic compounds found within plants as well as those naturally or synthetically derived and added to products.

As used herein, the term “batch number” is meant to encompass a the Oklahoma Medical Marijuana Authority defined unique numeric or alphanumeric identifier assigned prior to any testing to allow for inventory tracking.

This device is to be used to direct consumers as to which strain to use to achieve a desired result or effect.

The preferred embodiment of the device allows users to know which strain to consume to achieve a particular result or effect.

This device can be used in any environment.

Examples of alternative embodiments of the device are in any consumable form such as edible, topical or inhalable products

It is an object of the present invention to be simple for consumers to use.

It is an object of the present invention to direct consumers to achieve their desired affect.

Although the invention has been described in detail with reference to several embodiments additional variations and modifications exist within the scope of the spirit of the invention as described.

Although the disclosed invention has been described with reference to various exemplary embodiments, it is to be understood that these embodiments are merely illustrative of the principals and application of the present invention. Those having skill in the art would recognize that various modifications to the exemplary embodiments may be made, without departing from the scope of then invention.

Moreover, it should be understood that various features and/or characteristics of differing embodiments herein may be combined with one another. It is therefore to be understood that numerous modifications may be made to the illustrative embodiments and that other arrangements may be devised without departing from the scope of the invention.

Furthermore, other embodiments of the invention will be apparent to those skilled in the art from considerations of the specification and practice of the invention disclosed herein. It is intended that the specification and examples be considered as exemplary only, with a scope and spirit being indicated by the claims.

Finally, it is noted that, as used in this specification and the appended claims, the singular forms “a”, “an”, and “the,” include plural referents unless expressly and unequivocally limited to one referent, and vice versa. As used herein, the term “include” or “comprising” and its grammatical variants are intended to be non-limiting, such that recitation of an item or items is not to the exclusion of other like items that can be substituted or added to the recited item(s).

### DESCRIPTION OF THE DRAWINGS

### Brief Description of the Drawings

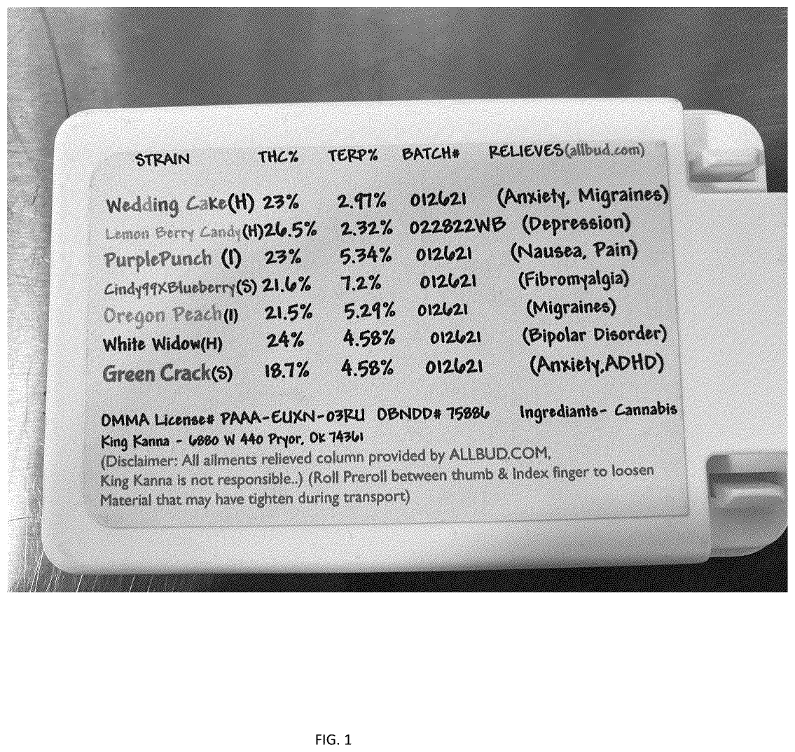

FIG. 1 shows an embodiment of the outer package portion of the product.

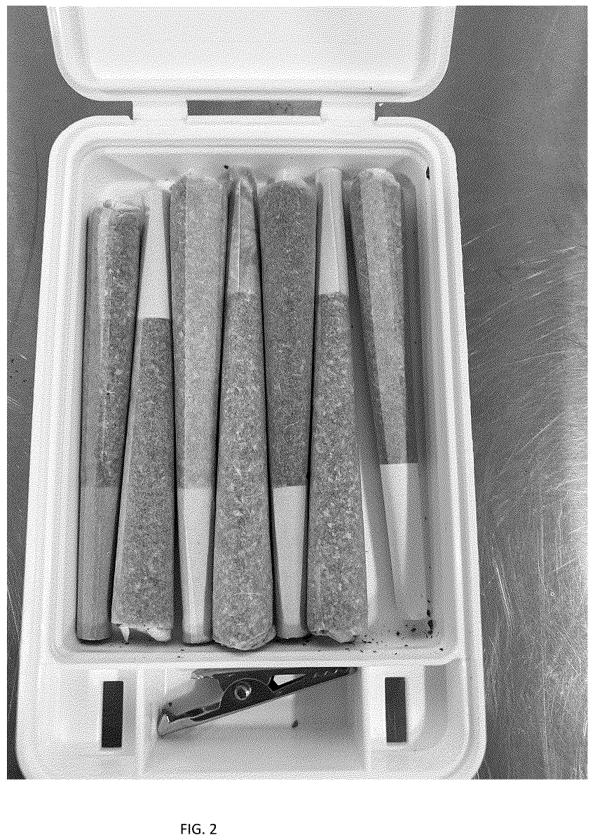

FIG. 2 shows the specific products inside of the outer package portion of the product.

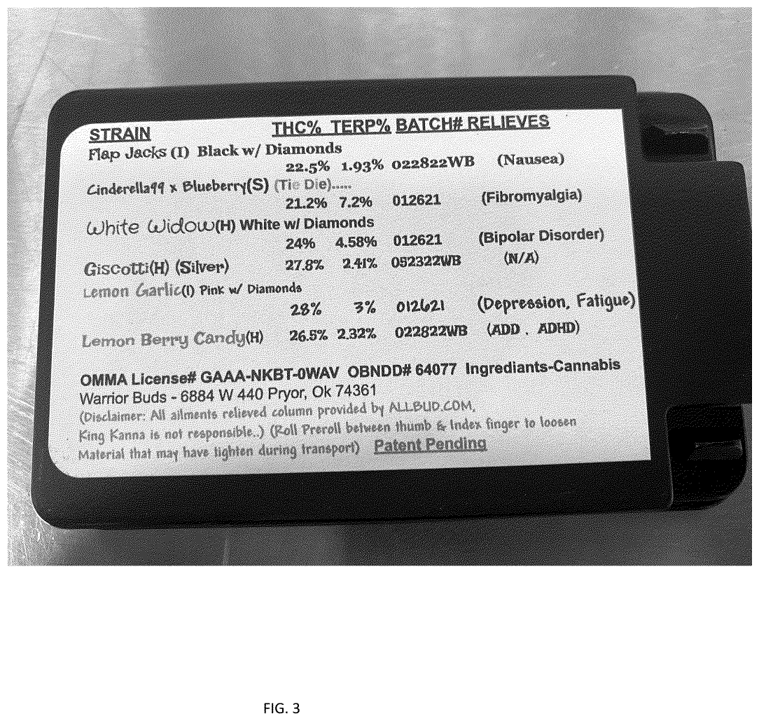

FIG. 3 shows another embodiment of the outer package portion of the product.

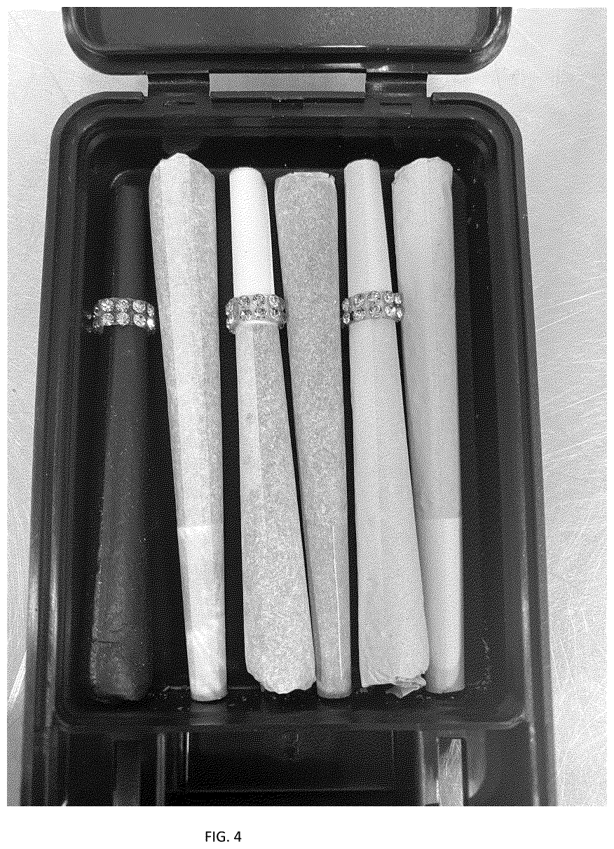

FIG. 4 shows the specific products inside of the outer package portion of the product.

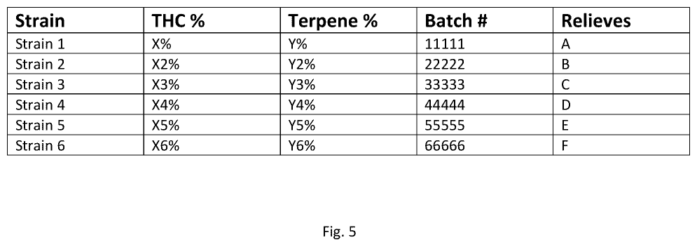

FIG. 5 shows a table of an example embodiment of the outer package portion of the labeling of the product.

### DETAILED DESCRIPTION OF THE DRAWINGS

FIG. 1 shows the outer package portion of the device featuring the strain name, all listed in a color specific font or listed visual characteristic to correspond to the specific color or visual characteristic of the strain specific product inside of the package.

FIG. 2 shows the color-coded or visually identifying strain specific products inside of the outer package portion.

FIG. 3 shows the outer package portion of the device featuring the strain name, all listed in a color specific font or listed visual characteristic to correspond to the specific color or visual characteristic of the strain specific product inside of the package.

FIG. 4 shows the color-coded or visually identifying strain specific products inside of the outer package portion.

FIG. 5 shows a table featuring an example embodiment of the main layout of the outer packaging label with the strain, percentages of active compounds of the products, as well as conditions relieved by the strain specific product.

What is claimed is:

1. A novel product comprising: at least two or more color-coded or distinguishable consumable cannabis containing products wherein each color identifies the particular type of cannabis strain within each said product.

2. The product of claim 1 wherein said cannabis containing products are contained within a single package.

3. The product of claim 1 wherein said cannabis containing products also contain another psychoactive substance to achieve an additional affect.

4. The product of claim 1 wherein said cannabis containing product are smokeable products.

5. The product of claim 1 wherein said cannabis containing products are edible products.

6. The product of claim 1 wherein said cannabis containing products are topical products.

7. A method of use of a novel product comprising: a user selects a color-coded or otherwise visually identified cannabis containing product from a single package to achieve a desired effect from a specific strain.
